(19) United States
(12) Patent Application Publication
(10) Pub. No.: US 2017/0020636 A1
(43) Pub. Date: Jan. 26, 2017

(54) System and method for robotic digital scanning of teeth

(71) Applicants: Hadi Akeel, San Ramon, CA (US); George Wong, San Ramon, CA (US)

(72) Inventors: Hadi Akeel, San Ramon, CA (US); George Wong, San Ramon, CA (US)

(21) Appl. No.: 15/130,269

(22) Filed: Apr. 15, 2016

Related U.S. Application Data
(60) Provisional application No. 62/148,322, filed on Apr. 16, 2015.

(51) Int. Cl. A61C 9/00; A61B 1/24; A61C 19/04; A61C 13/00; A61C 13/34; A61B 1/00; A61B 34/30

(52) U.S. Cl. CPC A61C 9/006; A61B 1/00009; A61B 1/00149; A61B 1/24; A61B 34/30; A61C 13/0004; A61C 13/34; A61C 19/04

(57) ABSTRACT

A method of scanning a patient's mouth using a robot to obtain a 3-D model of the patient's teeth. A database can contain a 3D model of a generic jaw geometry approximated in size by geometric parameters and at least one jaw scanning pattern. Geometric measurements of a patient's jaw are taken, then these are applied to the geometric parameters of the generic jaw. A predetermined scanning pattern is used to program the robot to scan the teeth with a scanner. The robot program is executed to move the scanner along the jaw scanning pattern at a fixed or variable distance from surfaces of the patient's jaw in sequences of coverage to the end of a scanning pattern to produce a three-dimensional (3D) model of the patient's teeth.

This application is related to, and claims priority from, U.S. Provisional Patent application No. 62/148,322 filed Apr. 16, 2015. Application 62/148,322 is hereby incorporated by reference in its entirety.

### BACKGROUND

Field of the Invention

The present invention relates generally to the field of scanning teeth to construct dental replacements and more particularly to a system and method for robotic digital scanning of teeth.

Description of the Problem

With modern digital imagery, dentists often perform optical scanning of teeth to generate a 3D image of the teeth that can be used to construct dental replacements such as crowns. This method replaces the traditional approach of making rubber molds of the teeth that duplicates the form of the teeth. The 3D digital images have all the dimensional data of the teeth and can be used to machine crowns on CNC milling machines, for example, or used to generate milling paths for a robot to prepare the tooth for a crown. A tooth cut according to a 3D image and a crown fabricated from the data of the same digital image will fit perfectly and result in long lasting prosthesis.

To generate a digital image, optical scanners such as the TrueDef Scanner, marketed by the 3 m company may be used and passed over and around the teeth to scan the teeth surface. The scanner projects a scanning beam on the surface of the teeth and collects images that contain 3D geometric data of the teeth. The scanning beam needs to reach all around each tooth and shines enough light on the surfaces that can be detected by the image detectors, images are then “stitched” together to form a seamless continuum of images representing the full complement of the teeth to be scanned. The device software includes mathematical subroutines that accommodates and corrects for repeated and discontinuous scanning of the surfaces. However, for an ideal scan, the distance between the light source of the scanner and the scanned surface is best if kept within an optimal range value specified for each scanner (for the 3M scanner it is about 3 to 12 mm). Minor variations can be tolerated, but major deviations may result on the loss of image continuity and the need for a new search or restart of the scanning process, a time consuming and wasteful task. Often, the scanning beam, manipulated by the dentist or dentist assistant, may miss some teeth regions or expose them inadequately to register their form within the image. The dentist must then rescan the missed surface at the right angle and exposure that allows the software to stitch the missing images to the rest of the scan. To generate a good scan the dentist usually observes an image of the scan on a video monitor and observes what surface are scanned properly and which surfaces are missed. This visual feedback, of course, helps the dentist generate an acceptable image suitable for follow up fabrication processes and the completion of a dental procedure. The shortcomings of the current scanning procedures are:
The dentist has to spend his valuable professional time doing a manual, repetitive task an inefficient and wasteful task
A human has difficulty, if at all, maintaining an optimum scanning distance or scanning angle at each surface on each tooth and often repeats the scanning moves several times before getting the right scan.
The dentist must observe a video monitor during the process of scanning that distracts him/her from maintaining the desired scanning parameters of distance an angular orientation for the scanning beam; the registration of the beam to the scanned images is often lost and the scanning repeated fully or partially.
Appreciable training and trial and error experience is needed to use the scanner and is often frustrating for beginners.
Visual monitoring of the scanned surfaces is needed and adds cost of a video monitor to the scanning equipment.

### SUMMARY OF THE INVENTION

The present invention includes a procedure for optical scanning teeth for dental procedures performed in a mouth cavity that use an optical scanner mounted on a robot. The robot or other processor can contain a database with a 3D model of a generic jaw geometry approximated in size by geometric parameters and one or more jaw scanning patterns. The steps are:

manually taking geometric measurements of a patient's jaw;

applying the geometric measurements to the geometric parameters of the generic jaw to define an approximate 3D geometry of the patient's jaw;

recalling a predetermined jaw scanning pattern from the database;

adjusting the jaw scanning pattern to match the approximate 3D geometry of the patient's jaw;

executing a robot program to move from a home position and introduce the scanner into a mouth cavity at a start point of the jaw scanning pattern;

manually initiating the scanner to scan the patient's jaw with the scanning beam according to the jaw scanning pattern;

continuing to execute the robot program to move the scanner along the jaw scanning pattern at a fixed or variable distance from surfaces of the patient's jaw in sequences of coverage to the end of a scanning pattern;

recording 3D images generated by the digital optical scanner;

retracting the scanner away from the mouth cavity to the home position.

Objectives of the present invention are to solve the shortcomings of the current procedures as follows:
Establish a repeatable structure for the scanning process that minimizes path variations from one scan to another.
Allow an approach that maintains optimum scanning parameters of beam distance, angle, and coverage during the process
Utilize the accuracy and consistency of robotic operations to automate the scan and relieve the dentist from menial and repetitive tasks
Can be performed with the patient seated at any posture for maximum comfort
Can be performed by dental office support personnel with minimal training
Minimize trial and error by scanning perfectly first time, every time
Eliminate the need for a video monitor for possible cost savings
Can be integrated into a robot controls system for additional cost savings.
Frees the dentist time to devote to more productive professional tasks.

### DESCRIPTION OF THE FIGURES

Attention is now directed to several drawings that illustrate features of the present invention:

FIG. 1 shows a top view of a first scanning pattern (A pattern).

FIG. 2 shows a side view of the scan in FIG. 1

FIG. 3 shows how scanning distance can be maintained with a spacer.

FIG. 4 shows a top view of a second scanning pattern (B pattern).

FIG. 5 shows inclined path angles.

FIG. 6 is a diagram of the relationship between the scanner and the teeth.

FIG. 7 is a diagram of a teaching tool.

FIG. 8 shows typical jaw parameters.

Several drawings have been presented to aid in understanding the present invention. The scope of the present invention is not limited to what is shown in the figures.

### DESCRIPTION OF THE PREFERRED EMBODIMENTS:

The invention utilizes an optical digital scanner attached to a multi-axes robot, preferably with six degrees of freedom, to manipulate the scanner inside the mouth cavity of the patient. A line representation of the application of the invention is shown in the opposite figure. Since the robot does not need access to the mouth cavity from above, such as with a dentist, the patient can be seated at a comfortable posture and not exposed to glaring lights of the scanner or overhead lighting as often experienced at a dentist's office

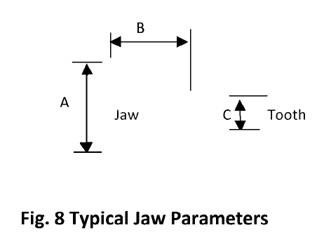

The robot may be programmed in one of two ways:
1. Guided by a programming pendant (teach pendant), points can be recorded along a desired robot path to guide the scanner in the vicinity of teeth top and sides according to predetermined scanning pattern. The points are recorded by the robot controller and, when played back, adjusted, verified, and considered satisfactory, are saved to the robot memory for use when needed. To help with accurate and expedient programming, a teaching aid may be utilized instead of an actual scanner. The teaching aid may take the form of an attachment to the scanner that touches the surface of teeth and separates the scanning surface by the optimum distance as shown in FIG. 3. An alternate method is the use of a scanning tool as shown in FIG. 7, which has a touch point separated from the robot with the same coordinates of the optimum scanning location of the real scanner. The touch point could be on a sphere of convenient non-obtrusive or diminutive size, possibly 1 mm to 5 mm diameter. When the touch point is guided to touch a point on a tooth and its location is recorded, it will represent a desired path point for the robot and used to generate the full robot path inside the mouth cavity. With this approach the path points are recorded relative to the robot geometric reference frame and the path can be traced directly by the robot without further referencing. However if the Jaw location is moved, such as when the patient moves an appreciable distance, additional referencing is necessary to execute the path in a common frame between the robot and the Jaw. Sensors may be used to sense the patient movement and adjust the robot path to accommodate patient movement in real time.
2. A 3D image of a “typical” mouth cavity may be recalled from a data base and scaled or dimensionally adjusted to the size of the patient's mouth cavity. The scaling parameters A, B and C are shown in FIG. 8 and may be used to scale the width, depth and height of the jaw relative to the “typical” mouth cavity. The scaled mouth cavity is then used as a virtual patient's mouth cavity and the robot programmed virtually, as is conventionally done with offline robot programming, to generate a virtual robot path that can be downloaded to the robot. The robot is then guided manually or by built-in sensors, such as vision sensors, to register to a correct point on the real teeth and the virtual teeth image and match their geometric coordinates. The scanning path can then be traced by the robot on the patient's mouth cavity as programmed virtually. A core element of the invention is the pattern of the scanning path. The pattern allows for full teeth coverage including inset cavities between teeth. The pattern is meant to be performed without visual feedback for verification. However, automated verification feedback, by built in sensors, may provide some desirable enhancements beyond basic scanning such as reducing scan time by skipping some sections of the pattern when the verification indicates their surfaces were scanned successfully in prior steps.

Different patterns may be used for obtaining a desired quality of scanning. Two preferred patterns are described herein. These may be considered as generic and can be used as is or modified for optimum results. The generic patterns are suitable for use in a majority of scans.

Depending on the geometry and dimensions of the patient's mouth cavity, the Dentist may use one of the preferred patterns directly or modify it and generate a new pattern and add it to a data base of available patterns for future use.

### Preferred Scanning Patterns

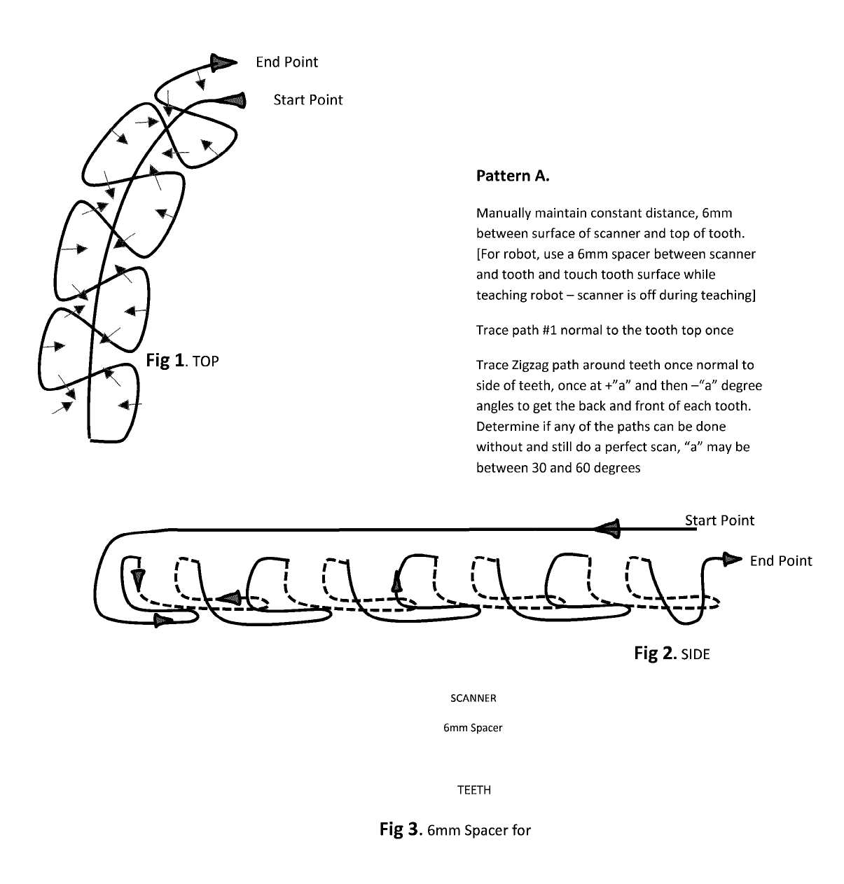

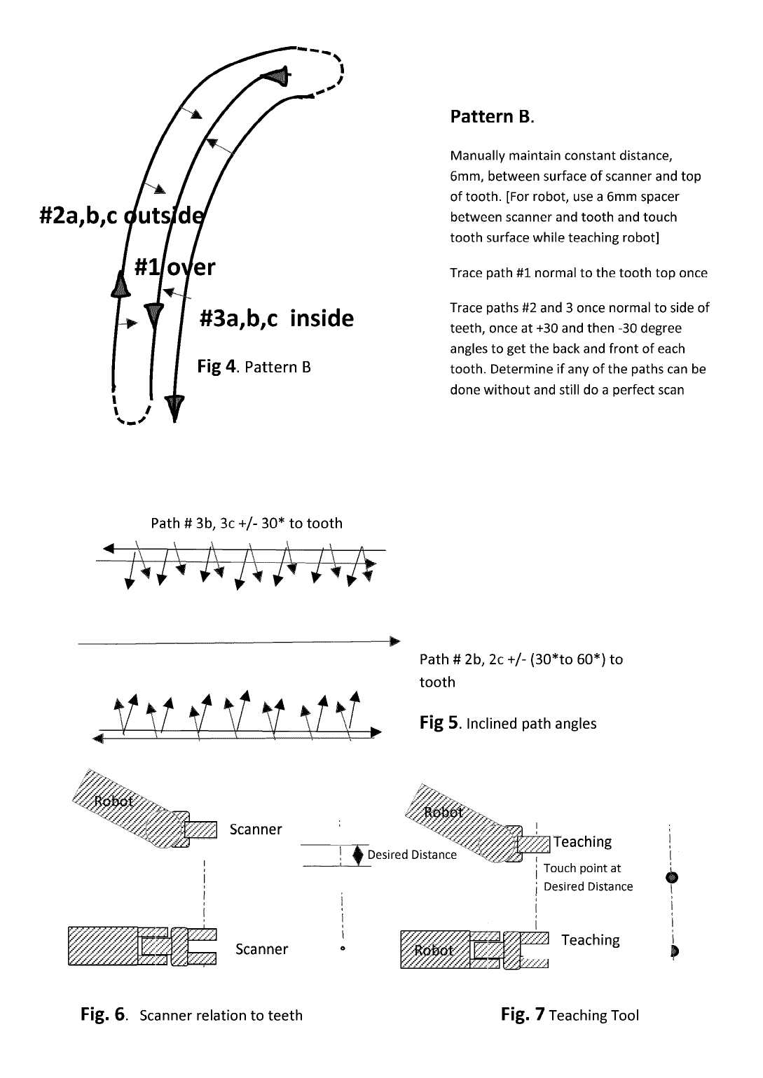

The preferred patterns are approximated by the following figures and structured to observe the following rules:
1. Scan the entire jaw (or segment of Jaw) over three surfaces, top, inside and outside normal to the teeth surfaces
2. Scan the sides with two passes, one inclined inwardly and the other inclined outwardly to cover insets between teeth from two directions as shown in FIG. 5.
3. Pattern A shown in FIGS. 1, 2 and 5 uses one sweeping stroke along the top of the teeth followed by reciprocating strokes progressively moving in the following sequence: outside up inclined in, top right, inside down inclined in, inside up inclined out, top left, outside down inclined out.
4. Pattern B, shown in FIGS. 4 and 5, uses sweeping strokes along the top and sides of the teeth in any sequence as follows preferably starting with top surface to register the scanner to the teeth then sweeping along the top from the back to the front; scan along the inside and outside surfaces in any sequence and in a continuous flow with strokes inclining the beam in normal, inward, and outward directions. The continuous flow of moving strokes may cause the scanned areas to overlap each other to obtain redundant areas and allow the software to stitch images together.
5. Both patterns A and B may be scanned with oscillating angular beam orientation to cover the contours of each unique tooth. Either the scanning wand is oscillated or, in some brands other than 3 m′s, a beam directing lens may oscillate the beam
6. Both patterns A and B may also scan in a closer or farther range to pick up surfaces of varying depth such as in the biting surface of a molar where the cusps and fossas of the teeth form a 3D shape of varying depths.
7. Variations of these patterns are possible and may be preferable in special cases of teeth irregularity or overlapping structures.
8. Some of the scanning strokes may occasionally be skipped to save time when full coverage is unnecessary.
9. The angle of inclination can be one in the range of 10 to 80 degrees from the normal direction and preferably in the range of 30 to 60 degrees for generic scans
10. Instead of a steady angle of inclination the robot can be programmed to oscillate the beam in a periodic motion with angular amplitudes, +/− a degrees, best suited for full scanning coverage where 10> a >80 degrees and 30 degrees being a recommended value for a typical application, with beam oscillations built into the robot path, two of the sweeping strokes, with fixed beam inclination angle, along the sides may be eliminated in some cases.

Several descriptions and illustrations have been presented to aid in understanding the present invention. One with skill in the art will realize that numerous changes and variations may be made without departing from the spirit of the invention. Each of these changes and variations is within the scope of the present invention.

We claim:

1. A procedure for optical scanning teeth for dental procedures performed in a mouth cavity comprising:
mounting an optical scanner having a scanning beam on a robot, the robot containing a processor with a memory, the memory storing a robot program that includes executable instructions for controlling the robot and containing a database including a 3D model of a generic jaw geometry approximated in size by geometric parameters and at least one jaw scanning pattern;
manually taking geometric measurements of a patient's jaw;
applying the geometric measurements to the geometric parameters of the generic jaw to define an approximate 3D geometry of the patient's jaw;
recalling a predetermined jaw scanning pattern from the database;
adjusting the jaw scanning pattern to match the approximate 3D geometry of the patient's jaw;
executing a robot program to move from a home position and introduce the scanner into a mouth cavity at a start point of the jaw scanning pattern;
manually initiating the scanner to scan the patient's jaw with the scanning beam according to the jaw scanning pattern;
continuing to execute the robot program to move the scanner along the jaw scanning pattern at in sequences of coverage to the end of a scanning pattern;
recording 3D images generated by the digital optical scanner;
retracting the scanner away from the mouth cavity to the home position.

2. The procedure of claim 1 wherein surfaces on the top and sides of teeth are scanned in a sequence of coverage with the scanning beam scanning at two angles of inclination to the surfaces.

3. The procedure of claim 2 wherein the angles of inclination are in a range of 10 to 80 degrees from a normal direction.

4. The procedure of claim 2 wherein the angles of inclination are in a range of 30 to 60 degrees from a normal direction.

5. The procedure of claim 1 wherein the geometric parameters are width of jaw at a back end, depth of jaw ends internal to mouth, and height of teeth.

6. The procedure of claim 1 wherein the generic jaw geometry is one of a size of an average jaw, typically medium size in a classification range of small, medium, and large.

7. The procedure of claim 1 wherein the step of adjusting includes the application of a software routine that scales the jaw scanning pattern to the size of the patient's jaw.

8. The procedure of claim 1 wherein the scanning beam is oscillated along the jaw scanning pattern.

9. The procedure of claim 8 wherein the scanning bean oscillates in a periodic motion with angular amplitudes being plus or minus a degrees, where 10>a >80 degrees.

10. The procedure of claim 8 wherein a is plus or minus 30 degrees.

11. The procedure of claim 1 wherein the jaw scanning pattern uses one sweeping stroke along the top of the teeth followed by reciprocating strokes progressively moving in a sequence comprising: (a) outside up inclined in, (b) top right; (c) inside down inclined in, (d) inside up inclined out, (e) top left, (f) outside down inclined out.

12. The procedure of claim 1 wherein the scanner scans in a closer or farther range to pick up surfaces of varying depth such as in a biting surface of a molar wherein cusps and fossas of the teeth form a 3D shape of varying depths.

13. The procedure of claim 1 wherein the jaw scanning pattern uses sweeping strokes along the top and sides of the teeth.

14. The procedure of claim 13 wherein the jaw scanning pattern starts with top surface to register the scanner to the teeth, then sweeps along the top from the back to the front, then scans along the inside and outside surfaces with strokes inclining the beam in normal, inward, and outward directions.

15. A procedure for optically scanning teeth in a patient's mouth with a scanner controlled by a robot comprising:
measuring a patient's jaw parameters;
applying these jaw parameters to a generic jaw model to produce a generic 3D model of the patient's mouth;
scanning the patient's mouth with a predetermined robot jaw scanning pattern creating a 3-D model of the teeth.

16. The procedure of claim 15 wherein the robot jaw scanning pattern uses one sweeping stroke along the top of the teeth followed by reciprocating strokes progressively moving in a sequence comprising: (a) outside up inclined in, (b) top right; (c) inside down inclined in, (d) inside up inclined out, (e) top left, (f) outside down inclined out.

17. The procedure of claim 15 wherein the scanning beam is oscillated along the jaw scanning pattern.

18. The procedure of claim 15 wherein surfaces on the top and sides of teeth are scanned in a sequence of coverage with the scanning beam scanning at two angles of inclination to the surfaces.
